(19) United States
(12) Patent Application Publication
(10) Pub. No.: US 2010/0189659 A1
(43) Pub. Date: Jul. 29, 2010

(54) DIAGNOSTIC SUBSTANCE AND METHOD FOR THE DIAGNOSIS OF PROSTATE DISEASES

(76) Inventors: Jens Fehre, Hausen (DE); Ralf Nanke, Neunkirchen am Brand (DE); Martin Stetter, Munchen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/665,904

(22) PCT Filed: Jun. 17, 2008
(86) PCT No.: PCT/EP08/57626

§ 371 Date: Dec. 21, 2009

(30) Foreign Application Priority Data
Jun. 21, 2007 (DE) 10 2007 028 659.9

(51) Int. Cl. A61K 49/16; A61P 13/08

(52) U.S. Cl. 424/9.6; 424/9.1

(57) ABSTRACT

A diagnostic substance for use in a method for diagnosing prostate diseases contains multiple different coupling molecules that respectively bind to signal molecules (target molecules) of a molecular surface structure of a pathological tissue that is associated with a prostate disease. A first of the coupling molecules specifically binds to signal molecules of cancer tissue, and a second of the coupling molecules specifically binds to signal molecules formed in inflamed tissue. First and second markers are respectively associated with the first and second coupling molecules, the first and second markers being detectable, and differentiated from each other, with a detection device.

### BACKGROUND OF THE INVENTION

1. Field of the Invention

The present invention concerns a diagnostic substance for application in a method for the diagnosis of prostate diseases, as well as such a method.

2. Description of the Prior Art

Given the occurrence of certain symptoms, or in the course of preventative measures, it is standard to give a patient a digital examination and to determine the PSA concentration in the blood. PSA is the abbreviation for prostate-specific antigen, a protein that is almost exclusively formed in the prostate. It is contained in prostate secretions and serves for the liquefaction of the semen. It also crosses over in small quantities into the blood and can be detected there with a laboratory test. If the PSA values exceed a specific value, the probability that a prostate carcinoma exists are increased. However, the cause may be a benign prostate disease, for instance benign prostate hyperplasia (BPH)—prostate enlargement—or prostatitis (prostate inflammation). The digital examination is less specific, such that multiple biopsies that are uncomfortable for the patient and that involve complex laboratory tests are required for a certain diagnosis.

### SUMMARY OF THE INVENTION

An object of the invention is to provide a diagnostic substance of the aforementioned type and a diagnostic method that allow detection of prostate diseases that is reliable and simple to implement, in particular a differentiation of prostate cancer from BPH and prostatitis.

A diagnostic substance according to the invention contains a molecule that specifically couples or binds (and therefore here is designated as a coupling molecule) to a molecular structure formed as a result of a specific disease in the prostate tissue. As used herein, “molecular structure” are individual molecules, molecular assemblies or molecular surface structures. A tissue with a specific pathological variation can be addressed with high specificity with the coupling molecule, which interacts with the molecular structure according to the key-lock principle, for example. The coupling molecules are provided with a marker so that an accumulation of coupling molecules in a specific pathologically altered tissue can be established. Based primarily on the fact that a complicated and (for the patient) uncomfortable biopsy is not required, the diagnostic substance or a diagnostic method implemented with it can be designed as an inexpensive screening test, for instance for the physician in private practice.

A coupling molecule that binds to a signal molecule formed by a pathological tissue is advantageously used. Signal molecules serve for intracellular communication, for example, and frequently accumulate in vascular endothelium, whereby they can easily be reached via the bloodstream. Different signal molecules are found in a prostate carcinoma than in prostatitis tissue, such that a selective addressing of this tissue with corresponding coupling molecules (advantageously with antibodies that bind very specifically) is possible. In the case of prostate carcinoma, the use of CEACAM1 antibodies as coupling molecules is particularly advantageous with regard to an early detection since CEACAM1 (which counts among the cell adhesion molecules) already forms in an early stage of the prostate carcinoma. A coupling molecule that binds to a signal molecule formed in an inflamed tissue is contained in the diagnostic substance for the diagnosis of prostatitis. Here as well corresponding antibodies are conceivable as coupling molecules. Selections (which are among the cell adhesion molecules) already form in the vascular endothelium in inflamed tissues at an early stage of inflammation. An early prostatitis diagnosis is therefore possible with a coupling molecule binding to selection, in particular with the selection ligand sialyl Lewis X. A cell adhesion molecule that likewise forms in an early inflammation stage is integrin, which can be addressed with integrin ligands as coupling molecules, for example. In general, other molecules (for example aptamers or anticalins) can also be used, for example instead of antibodies that often can only be produced or obtained with difficulty. Aptamers are selectively binding RNA chains. Anticalins are synthetic polypeptides. By corresponding protein design, they can be designed so that the antibodies possess corresponding binding properties.

A number of medically tested possibilities are provided for the detection of the coupling molecules accumulating in a diseased tissue. For example, magnetic or magnetizable particles that are detectable with the use of magnetic field sensors can be used as markers. Markers can also be used that absorb electromagnetic waves, for instance visible light or x-rays, and can be detected using their absorption spectrum for example. If the diagnostic substance contains different coupling molecules, i.e. coupling molecules respectively accumulating in a specific pathological tissue, the respective markers must possess different absorption spectra so that they can be differentiated with the detection device and different disease focii can be detected. Marker dyes that absorb in the near-infrared are particularly preferred. Human and animal tissue is particularly permeable to NIR, whereby regions situated deeper in the tissue can be reached. Fluorescent dyes are particularly preferred since these normally absorb in a wavelength range different from their fluorescence spectrum so that the fluorescence light that they emit can easily be filtered out from the excitation light. For the reasons cited above, a fluorescent dye whose absorption and fluorescence spectrum lie in the near-infrared is advantageous.

To implement a diagnosis, the diagnostic substance (which advantageously is administered intravenously) is supplied to the target area via the bloodstream, and an accumulation of marked coupling molecules is established with the aid of a detection device positioned in or on the body. The detection device is advantageously integrated into a rectal probe. Examinations targeting the diagnosis of a specific disease are respectively conducted in chronological order, wherein the presence of a marker accumulation is tested with the aid of the detection device after administration of a specific diagnostic substance. Particularly with regard to examinations conducted within the scope of screening tests, it is advantageous when a diagnostic substance is administered that addresses different diseases, which diagnostic substance thus contains the corresponding coupling molecules and markers individualizing these. A detection device used in this case is designed to detect the different markers with sufficient discriminatory power. The presence of a specific disease, for example of a prostate carcinoma, can thus be reliably established with a single test.

### BRIEF DESCRIPTION OF THE DRAWINGS

FIG. 1 schematically illustrates the formation of signal molecules in prostate tissue.

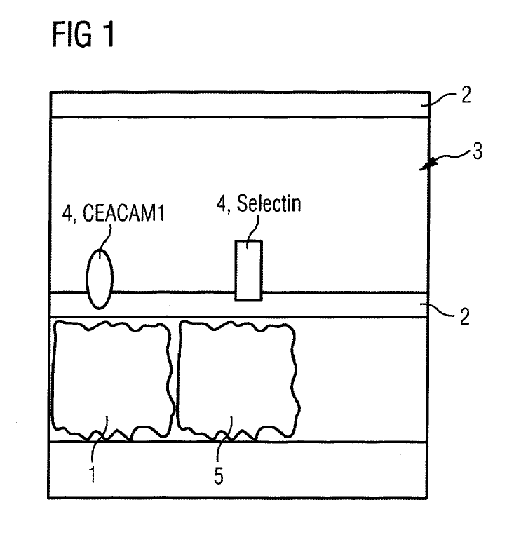

FIG. 2 schematically illustrates the implementation of a differential diagnosis in accordance with the present invention.

### DESCRIPTION OF THE PREFERRED EMBODIMENTS

As in other body tissues as well, signal molecules 4 form in the wall 2 of a blood vessel 3 in the prostate given the presence of a carcinoma 1. In the case of a carcinoma 1, these are for example CEACAM1 molecules. In the case of a prostatitis tissue 5, selectin molecules likewise form in the wall 2 or, respectively, an endothelium of blood vessels 3 that line said wall 2. The presence of the cited signal molecules 4 is now utilized for specific detection of this tissue in that coupling molecules 7 provided with markers 6 and specifically binding to the cited signal molecules 4 are used. In the case of prostate carcinoma 1, for example, a CEACAM1 antibody is used. The selectin ligand sialyl Lewis X that binds to selectin is used as a coupling molecule 7 in the case of prostatitis tissue 5. The markers 6 are generally molecules or particles that can be detected with the aid of a detection device 8. For example, the markers are fluorescent dyes whose absorption and fluorescence spectrum lies in the near-infrared, thus in a wavelength range from 750 to 2000 nm, for instance. Human and animal tissue is particularly transmissive in the near-infrared range, such that even tissue layers situated deeper can be reached.

The detection device 8 is, for example, integrated into a rectal probe 9 and comprises at least one illumination device 10 (for example in the form of an LED); a receiver element 11 sensitive to the respective fluorescent light; and a readout unit that transduces the optical signals into an electrical signal 13.

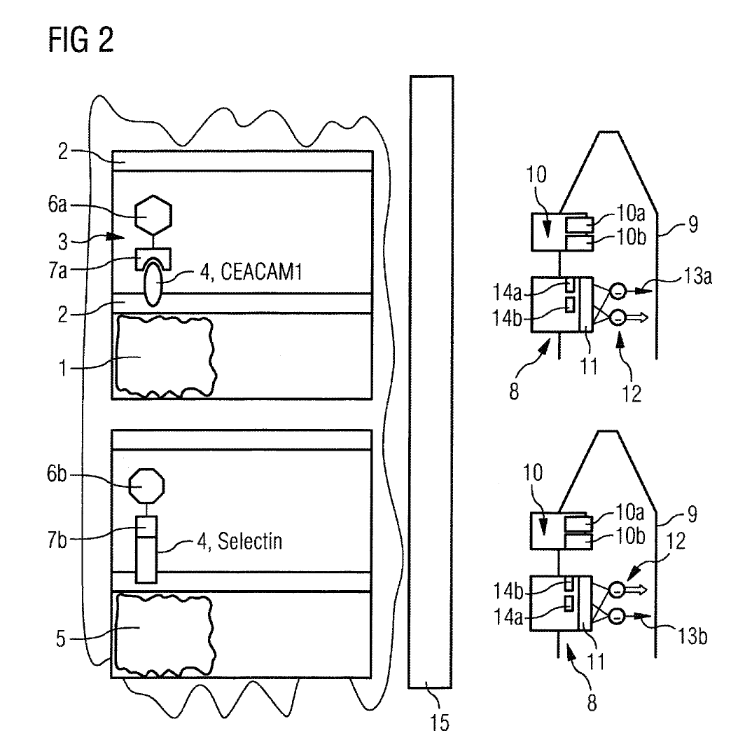

A method is indicated in FIG. 2 in which a diagnostic substance is administered that contains different coupling molecules, namely that that bind to CEACAM1 if prostate carcinoma and those that bind to selectin of prostatitis tissue, for example CACAM1 [sic] antibodies and sialyl Lewis X. The coupling molecules 7, 7a, 7b are respectively connected with markers 8a, 8b, namely with at least one fluorescence dye molecule, wherein these exhibit different fluorescence spectra so that they can be differentiated from one another and a simultaneous detection is thereby possible. The intestinal wall 15 present between the prostate and the rectal probe 9 is penetrated both by the excitation light and the fluorescence light without difficulty. For example, a dye molecule that fluoresces in the short-wave range is used as a marker 8a for cancer detection. Here the fluorescent dye NIR-1 with an absorption maximum at 755 nm and a fluorescence maximum at 790 nm is considered, for example. A dye fluorescing in a longer-wave range, for example indocyanine green with an absorption maximum at 800 nm and a fluorescence maximum at 830 nm, is used for prostatitis detection. The rectal probe 9 or, respectively, another detection device contains corresponding narrowband LEDs 10a, 10b as an illumination device and a likewise narrowband receiver element 11. The LED 10a is matched to the marker 6a of the cancer-sensitive coupling molecule 7a; the LED 10b is matched to the marker 6 of the prostatitis-sensitive coupling molecule 7b. Upstream of these are possibly narrowband filters 14a, 14b that are matched to the fluorescent light of the marker 6a or, respectively, 6b, i.e. absorb wavelengths lying outside of the respective fluorescence spectrum. Depending on what clinical picture is present, a signal 13a or, respectively, 13b is generated which indicates a prostate carcinoma or a prostatitis. The respective other coupling molecules are distributed in the body and ultimately excreted. A BPH is diagnosed if, in spite of an enlarged prostate, neither cancer-sensitive nor prostatitis-sensitive coupling molecules accumulate. For measurement reasons, it can be advantageous to provide a detection unit as a reference (not shown) which measures the absolute light intensity of the background. A more precise readout result can be achieved with a detection unit (not shown) that is designed as an imaging unit. Prevalent methods such as laser scanning methods or camera-based methods (the latter known from fluorescence angiography of the eye) are thereby used.

Although modifications and changes may be suggested by those skilled in the art, it is the intention of the inventors to embody within the patent warranted heron all changes and modifications as reasonably and properly come within the scope of their contribution to the art.

We claim as our invention:

1.-19. (canceled)

20. A diagnostic substance for diagnosis of prostate diseases, comprising:
a plurality of different coupling molecules that each binds to signal molecules of a molecular surface structure of a pathological tissue associated with prostate diseases;
a first of said coupling molecules specifically binding to signal molecules of cancer tissue;
a second of said coupling molecules specifically binding to signal molecules formed in inflamed tissue; and
a first marker associated with said first of said coupling molecules and a second marker associated with said second of said coupling molecules, each of said first and second markers having a characteristic allowing detection of said first and second markers with a detection device and, based on said detection, also allowing said first and second markers to be differentiated from each other.

21. A diagnostic substance as claimed in claim 20 wherein at least one of said coupling molecules is an antibody.

22. A diagnostic substance as claimed in claim 21 wherein said antibody is CEACAM1.

23. A diagnostic substance as claimed in claim 20 wherein at least one of said coupling molecules is a ligand selected from the group consisting of a selectin ligand and integrin ligand.

24. A diagnostic substance as claimed in claim 23 wherein said second of said coupling molecules is sialyl Lewis X.

25. A diagnostic substance as claimed in claim 20 wherein at least one of said coupling molecules is aptamer.

26. A diagnostic substance as claimed in claim 20 wherein at least one of said coupling molecules is anticalin.

27. A diagnostic substance as claimed in claim 20 wherein at least one of said first and second markers is a marker that absorbs electromagnetic waves.

28. A diagnostic substance as claimed in claim 27 wherein at least one of said first and second markers that absorbs electromagnetic waves absorbs electromagnetic waves in a near-infrared portion of the electromagnetic spectrum.

29. A diagnostic substance as claimed in claim 28 wherein said at least one of said first and second markers is a fluorescent dye.

30. A diagnostic substance as claimed in claim 29 wherein said fluorescent dye has both an absorption spectrum and a fluorescent spectrum in said near-infrared range.

31. A diagnostic substance as claimed in claim 30 wherein said first and second markers respectively exhibit different fluorescence spectra allowing said first and second markers to be differentiated from each other by said detection device.

32. A diagnostic substance as claimed in claim 31 wherein said first marker has a fluorescence spectrum with a maximum at 790 nm.

33. A diagnostic substance as claimed in claim 32 wherein said second marker has a fluorescence spectrum with a maximum at 830 nm.

34. A diagnostic substance as claimed in claim 31 wherein said first marker is NIR-1 and wherein said second marker is indocyanine green.

35. A method for diagnosing prostate diseases comprising the steps of:
administering a diagnostic substance to a patient, said diagnostic substance comprising a plurality of different coupling molecules that each bind to signal molecules of a molecular surface structure of a pathological tissue associated with prostate diseases, a first of said coupling molecules specifically binding to signal molecules of cancer tissue, a second of said coupling molecules specifically binding to signal molecules formed in inflamed tissue, and a first marker associated with said first of said coupling molecules and a second marker associated with said second of said coupling molecules, each of said first and second markers having a characteristic allowing detection of said first and second markers with a detection device and, based on said detection, also allowing said first and second markers to be differentiated from each other; and.
detecting said first and second markers with said detection device and differentiating said markers, and thus also differentiating said first of said coupling molecules and said second of said coupling molecules, with said detection device.

36. A method as claimed in claim 35 comprising administering a plurality of different diagnostic substances to the patient, with each of said different diagnostic substances comprising different coupling molecules that specifically bind to target molecules of different prostate-related diseases.

37. A method as claimed in claim 36 comprising detecting said plurality of different coupling molecules with a single detection device that is capable of detecting all markers in all of said different diagnostic substances.

38. A method as claimed in claim 35 comprising detecting said first and second markers with said detection device integrated into a rectal probe.
